(19) United States
(12) Patent Application Publication
(10) Pub. No.: US 2026/0007584 A1
(43) Pub. Date: Jan. 8, 2026

(54) COMPOSITION FOR USE IN CONCEALING, HEALING, AND/OR MOISTURING A BRUISE

(71) Applicants: John Bartolone, Bridgeport, CT (US); Vladimir Ratushny, Manchester-By-The-Sea, MA (US); Anar Mikailov, Victor, NY (US)

(72) Inventors: John Bartolone, Bridgeport, CT (US); Vladimir Ratushny, Manchester-By-The-Sea, MA (US); Anar Mikailov, Victor, NY (US)

(21) Appl. No.: 19/257,635

(22) Filed: Jul. 2, 2025

Related U.S. Application Data
(60) Provisional application No. 63/668,076, filed on Jul. 5, 2024.

(51) Int. Cl. A61K 8/29; A61K 8/25; A61K 8/26; A61K 8/34; A61K 8/37; A61K 8/44; A61K 8/67; A61K 8/9789; A61Q 1/02; A61Q 19/00

(52) U.S. Cl. CPC A61K 8/29; A61K 8/25; A61K 8/26; A61K 8/345; A61K 8/347; A61K 8/375; A61K 8/44; A61K 8/671; A61K 8/676; A61K 8/678; A61K 8/9789; A61Q 1/02; A61Q 19/007; A61K 2800/42; A61K 2800/5922; A61K 2800/805

(57) ABSTRACT

The invention is a composition that can be used to effectively treat or heal bruises that form from injury and/or senile purpura. Specifically, the disclosed composition beneficially heals, moisturizes, and conceals bruised skin. The invention also includes methods of making and using the disclosed composition to aid in wound healing, concealment, and moisturizing.

### CROSS-REFERENCE TO RELATED APPLICATIONS

This application claims priority to U.S. Provisional Patent Application No. 63/668,076, filed Jul. 5, 2025, the entire content of which is incorporated by reference herein.

### TECHNICAL FIELD

The presently disclosed subject matter relates to a composition for use with bruise healing, concealing, and/or moisturizing.

### BACKGROUND

Bruises often form when small blood vessels (capillaries) near the skin's surface are broken by the impact of a blow or trauma. When this happens, blood leaks out of the vessels and initially appears as a dark red mark on the surface of the skin. Eventually, the body reabsorbs the blood, and the mark disappears. As people age, the skin becomes thinner and loses some of the protective fatty layer that helps protect blood vessels from injury (often referred to as senile or actinic purpura). The primary symptom of actinic purpura is large, purplish red bruises that are commonly located on the backs of the hands or the forearm. The bruises typically last between one and three weeks before fading. The bruises fade to brown instead of having the typical coloration of a fading bruise, which may look blue, green, or yellow at different healing points.

For women, actinic bruising on the back of hands, outer forearms, and legs is visibly undesirable. Women try to cover these areas with clothing or gloves. Whether a result of injury or actinic purpura, finding solutions that can heal and or conceal these bruises is of high interest to women of all ages. Despite being a benign condition, the continual development of purpura lesions in afflicted women is frequently a source of significant visual and social concern.

Around 12 percent of people develop senile purpura after the age of 50, and up to 30 percent after the age of 75. Therefore, a rather large number of people suffer from purpura. As the oldest population of people aged 60 or older is also the largest growing population, the need to find simple, acceptable, effective and quick remedy solutions that can partially or fully conceal purpura will continue to be of high importance. It would therefore be desirable to provide a composition that can effectively heal, moisturize, and conceal a bruise.

### SUMMARY

In some embodiments, the presently disclosed subject matter is directed to a composition comprising one or more skin safe colored materials that deposit on the skin (e.g., are topically applied) and partially to fully color conceal, color correct, or both color conceal and color correct bruises in various healing phases. “Color conceal” refers to the characteristic of balancing out skin discolorations using green, peach, pink, purple, yellow, etc. to counteract a bruise or purpura. “Color correct” refers to the characteristic of neutralizing skin discoloration. For example, green neutralizes redness, peach and orange counteract blue tones, yellow combats purple and blue hues, lavender brightens the skin, and pink conceals dark areas and brightens skin in some embodiments. “Partially” includes about 25-95 percent (e.g., at least/no more than about 25, 30, 35, 40, 45, 50, 55, 60, 65, 70, 75, 80, 85, 90, or 95 percent). “Fully” refers to an amount of about 100 percent (e.g., the bruise or purpura is not visible on the skin). The composition also includes one or more bruise healing materials (e.g., materials capable of healing or promoting healing of bruises, senile purpura, and/or solar purpura). In some embodiments, purpura is considered to be a type of bruise.

In some embodiments, the composition further comprises a moisturizing base.

In some embodiments, the skin safe colored materials are selected from: titanium dioxide, colored iron oxides, mica, chalks, clays, or combinations thereof and/or the bruise healing materials are selected from: Arnica, Vitamin K, Vitamin C, Vitamin E, Tranexamic acid, retinol, bakuchiol, retinyl palmitate, or combinations thereof.

In some embodiments, the moisturizing base comprises glycerin, caprylic triglycerides, capric triglycerides, coconut oil, olive oil, vegetable oils, dimethicone, steric acid, butylene glycol, cetyl alcohol, or combinations thereof.

In some embodiments, the skin safe colored materials comprise a yellow, brown, or pink tint that is configured to visually conceal or color correct a bruise partially or fully without the need of an additional colored material.

In some embodiments, the skin safe colored materials are selected from about 1-99 weight percent of titanium dioxide, 1-99 weight percent of yellow iron oxide, 1-99 weight percent of pink iron oxides, 1-99 weight percent of red iron oxides, 1-99 weight percent of mica, 1-99 weight percent chalk, and 1-99 weight percent clay.

In some embodiments, the composition is configured to partially or fully conceal a skin bruise after one or more applications.

In some embodiments, the composition is color transfer resistant.

In some embodiments, the composition is compatible and covered using a skin color matched foundation or makeup. Stated another way, the composition can be covered by applying foundation or makeup to the skin, over the disclosed composition (e.g., after the composition has dried on the skin in some embodiments).

In some embodiments, the composition includes one or more depigmenting materials. The term “depigmenting material” refers to a material that can inhibit or reduce melanin synthesis in melanocytes and thus lighten the skin. Suitable depigmenting agents can include (but are not limited to) one or more of fluocinolone/hydroquinone/tretinoin, hydroquinone, dilauryl thiodipropionate, Kojic acid, hydroquinone, salicylic acid, and monobenzone. The depigmenting agent can be present in the composition in an amount of about 5-50 weight percent (e.g., at least/no more than about 5, 10, 15, 20, 25, 30, 35, 40, 45, or 50 weight percent).

In some embodiments, the composition can include one or more pain reducing or relieving ingredients. Suitable agents to reduce or relieve pain can include salicylates, menthol, camphor, lidocaine, and the like in an amount of about 5-50 weight percent (e.g., at least/no more than about 5, 10, 15, 20, 25, 30, 35, 40, 45, or 50 weight percent).

In some embodiments, the presently disclosed subject matter is directed to a method of concealing a bruise (e.g., visually concealing a bruise fully or partially) using the disclosed composition. Particularly, the method comprising applying the composition to a bruise (e.g., covering the colored portion of the bruise) and allowing the composition to dry, whereby the bruise is concealed and healing is promoted.

In some embodiments, the applying is facilitated using a sponge pad, a spot applicator, or both. A sponge pad can be a portion of material (such as sponge or gauze) that can be used to absorb an amount of the composition and transfer the composition to a desired location on the user's skin (e.g., in the area of a bruise). A spot applicator is any type of tool used to apply a composition (e.g., a wand).

### BRIEF DESCRIPTION OF THE DRAWINGS

FIG. 1 is a photograph of an untreated bruise on a subject in accordance with some embodiments of the presently disclosed subject matter.

FIG. 2 is a photograph of the bruise of FIG. 1 after a single light application of the disclosed composition in accordance with some embodiments of the presently disclosed subject matter.

FIG. 3 is a photograph of the bruise of FIG. 1 after a second light treatment of the disclosed composition has been applied in accordance with some embodiments of the presently disclosed subject matter.

FIG. 4 is a photograph of a bruise prior to treatment in accordance with some embodiments of the presently disclosed subject matter.

FIG. 5 is a photograph of the bruise of FIG. 4 immediately after application of the disclosed composition in accordance with some embodiments of the presently disclosed subject matter.

FIG. 6 is a photograph of the bruise of FIG. 5 after the disclosed composition has dried after being applied over the area of the bruise in accordance with some embodiments of the presently disclosed subject matter.

### DETAILED DESCRIPTION

The presently disclosed subject matter is introduced with sufficient details to provide an understanding of one or more particular embodiments of broader inventive subject matters. The descriptions expound upon and exemplify features of those embodiments without limiting the inventive subject matters to the explicitly described embodiments and features. Considerations in view of these descriptions will likely give rise to additional and similar embodiments and features without departing from the scope of the presently disclosed subject matter.

Unless defined otherwise, all technical and scientific terms used herein have the same meaning as commonly understood to one of ordinary skill in the art to which the presently disclosed subject matter pertains. Although any methods, devices, and materials similar or equivalent to those described herein can be used in the practice or testing of the presently disclosed subject matter, representative methods, devices, and materials are now described.

Following long-standing patent law convention, the terms “a”, “an”, and “the” refer to “one or more” when used in the subject specification, including the claims. Thus, for example, reference to “a device” can include a plurality of such devices, and so forth.

Unless otherwise indicated, all numbers expressing quantities of components, conditions, and so forth used in the specification and claims are to be understood as being modified in all instances by the term “about”. Accordingly, unless indicated to the contrary, the numerical parameters set forth in the instant specification and attached claims are approximations that can vary depending upon the desired properties sought to be obtained by the presently disclosed subject matter.

As used herein, the term “about”, when referring to a value or to an amount of mass, weight, time, volume, concentration, and/or percentage can encompass variations of, in some embodiments +/−20%, in some embodiments +/−10%, in some embodiments +/−5%, in some embodiments +/−1%, in some embodiments +/−0.5%, and in some embodiments +/−0.1%, from the specified amount, as such variations are appropriate in the disclosed packages and methods.

The presently disclosed subject matter is directed to an improved composition that can be used to effectively treat and/or heal bruises that form from injury and/or senile purpura. “Treating” refers to the inhibiting or amelioration of a symptom of a bruise and/or purpura. “Healing” refers to eradication, reduction, amelioration, or reversal of a sign or symptom of a bruise or purpura. The term “bruise” refers to a type of hematoma of tissue characterized by localized bleeding that extravasates into the surrounding interstitial tissues. Bruises occur when blood vessels are damaged or broken due to an impact on the skin. The bruise results from the body's response to the injury as well as blood leakage from the injured blood vessels into the tissue.

In some embodiments, the term “bruise” can include senile purpura and/or polar purpura. The term “senile purpura” refers to a skin condition characterized by large (e.g., 1-5 centimeters in length) and sharply outlined dark purplish red ecchymoses that appear on the skin. The term “solar purpura” refers to a skin condition characterized by purple spots or bruises when small blood vessels leak under the skin, commonly caused by excessive sunlight exposure. As discussed in detail below, the disclosed composition beneficially heals, moisturizes, and/or conceals the bruised skin.

Most conventional skincare products in the marketplace today claim to treat or heal bruises that form from an injury or senile purpura using various biologically active ingredients. Specifically, the ingredients can reduce the inflammation and swelling caused by the injury or impact, strengthen the blood vessels (particularly of the purpura type), and improve the dermal cushion (referred to as “collagen”). Other prior art remedies include breaking down the associated clot and increasing blood flow via vasodilation. However, none of the conventional remedies and/or treatments provide an immediate solution to help conceal the bruise. Further, none of the prior art products provide the dual benefits of both concealing and healing the noted bruises.

Makeup protocols have conventionally been developed to help conceal bruises. Typically, the makeup includes several products including a foundation, concealer, and one of several color correctors. However, various implements are required to successfully apply and distribute the makeup with the hope that the individual has the time and understanding to properly combine these products to achieve the desired results. The sophistication of these practices may suggest a makeup artist is needed in many embodiments.

As noted above, the disclosed composition provides multiple benefits to the user. Specifically, the composition can conceal, moisturize, and/or heal bruised skin. The term “conceal” includes the characteristic of being hidden or removed from visual observation. The term “moisturize” refers to adding or restoring moisture to the skin. The term “heal” can include repairing of damaged tissues in the body.

Particularly, the disclosed composition comprises one or more skin safe colored materials that deposit on the skin to partially or fully color conceal, color correct, or both color conceal and color correct bruises in various healing phases. The term “skin safe” refers to a material that does not cause adverse effect when in contact with the skin of a user (e.g., irritation-free, rash-free, and the like). The term “colored materials” refers to any of a wide variety of materials that include a pigment. In some embodiments, the colored materials can be selected to match a user's skin tone. Any suitable skin safe colored material(s) can be used. For example, suitable materials can be selected from titanium dioxide, colored iron oxides, mica, chalk, clay, or combinations thereof.

Titanium dioxide is the inorganic compound derived from titanium with the chemical formula TiO₂ and includes all phases of titanium, including the anatase, rutile, and brookite phases, in all proportions.

Colored iron oxide includes any oxide or hydroxide of iron (e.g., FeO, Fe2O3, Fe3O4) or any combination thereof.

“Mica” refers to any of a group of hydrous aluminum silicate minerals. Mica can be, for example, sheet mica, scrap mica or flake mica, as exemplified by muscovite, biotite or phlogopite type micas. Many micas can be described generally by the chemical formula X₁Y₂₋₃Z₄O₁₀(OH, F)₂, in which: X is potassium (K), sodium (Na), calcium (Ca), hydronium (H₃O), ammonium (NH₄), Ba, Rb, or Cs; Y is Al, Mg, Fe, Mn, Cr, Ti, Li, V, or Zn; and Z is generally Si, Al, Fe³⁺ or Ti.

Chalk is a fine-textured, earthy type of limestone distinguished by its light color, softness, and high porosity. Chalk is composed mostly of tiny fragments of calcite shells or skeletons of plankton, such as foraminifera or coccolithophores.

The term “clay” refers to a crystalline form of hydrated aluminum silicate, such as (but not limited to) one or more types of clay, including, but not limited to kaolinite, montmorillonite/smectite, illite, chlorite, and any mixture thereof.

In some embodiments, the skin safe colored materials comprise a yellow, brown, and/or pink tint that is configured to visually conceal or color correct a bruise partially or fully without the need of an additional colored material.

The disclosed composition further includes one or more bruise healing materials. The term “bruise healing material” refers to a material or combination of materials that aid in bruise healing, such as by reducing the time of skin discoloration. The bruise healing materials can have any suitable mechanism of action, such as stimulation of blood flow, reduction of skin swelling, reducing inflammation, strengthening blood vessels, reducing leaking of blood under the skin, etc.

Suitable bruise healing materials can include (but are not limited to) one or more of the following: arnica, vitamin K, vitamin C, vitamin E, tranexamic acid, retinol, bakuchiol, or retinyl palmitate.

Arnica includes a genus of perennial, herbaceous plants in the sunflower family (Asteraceae). Arnica has been used to soothe muscle aches, reduce inflammation, and heal wounds. Arnica is typically applied to the skin as a cream, ointment, or tincture.

Vitamin K is a family of structurally similar, fat-soluble vitamers found in foods and marketed as dietary supplements. The human body requires vitamin K for post-synthesis modification of certain proteins that are required for blood coagulation or for controlling binding of calcium in bones and other tissues. Chemically, the vitamin K family comprises 2-methyl-1,4-naphthoquinone (3-) derivatives.

Vitamin C (also known as ascorbic acid and ascorbate) is a water-soluble vitamin found in citrus and other fruits, berries, and vegetables. Vitamin C is an essential nutrient involved in the repair of tissue, the formation of collagen, and the enzymatic production of certain neurotransmitters. It is required for the functioning of several enzymes and is important for immune system function. It also functions as an antioxidant.

Vitamin E is a group of eight fat soluble compounds that include four tocopherols and four tocotrienols. Vitamin E is a fat-soluble antioxidant that may help protect cell membranes from reactive oxygen species.

Tranexamic acid is a synthetic analog of the amino acid lysine. It serves as an antifibrinolytic by reversibly binding four to five lysine receptor sites on plasminogen. This decreases the conversion of plasminogen to plasmin, preventing fibrin degradation and preserving the framework of fibrin's matrix structure. Tranexamic acid is commonly used to treat or prevent excessive blood loss from major trauma, postpartum bleeding, surgery, tooth removal, nosebleeds, and heavy menstruation.

Retinol (also called vitamin A1) is a fat-soluble vitamin in the vitamin A family that is found in food and used as a dietary supplement. Retinol or other forms of vitamin A are needed for vision, cellular development, maintenance of skin and mucous membranes, immune function and reproductive development.

Bakuchiol is a meroterpenoid (a chemical compound having a partial terpenoid structure) in the class terpenophenol. Bakuchiol possesses antioxidant, anti-inflammatory, and antibacterial properties.

Retinyl palmitate is (or vitamin A palmitate) is the ester of retinol and palmitic acid, with the formula C₃₆H₆₀O₂. Retinyl palmitate is used as an antioxidant and a source of vitamin A.

Thus, in some embodiments, the disclosed composition can comprise about 1-99 weight percent skin safe colored materials and about 99-1 weight percent bruise healing materials. Accordingly, the composition can comprise at least about (or no more than about) 1, 5, 10, 15, 20, 25, 30, 35, 40, 45, 50, 55, 60, 65, 70, 75, 80, 85, 90, 95, or 99 weight percent skin safe colored material(s), based on the total weight of the composition. The composition can further comprise at least about (or no more than about) 1, 5, 10, 15, 20, 25, 30, 35, 40, 45, 50, 55, 60, 65, 70, 75, 80, 85, 90, 95, or 99 weight percent bruise healing material(s), based on the total weight of the composition.

Optionally, the disclosed composition further includes a moisturizing base. The term “moisturizing base” broadly includes a material or materials that supply moisture to the skin and/or block the evaporation of moisture to maintain the skin's flexibility. Materials suitable for use as a moisturizing base can include (but are not limited to) glycerin, caprylic triglyceride, capric triglyceride, coconut oil, olive oil, vegetable oil, dimethicone, steric acid, butylene glycol, cetyl alcohol, or combinations thereof.

Glycerin is a simple triol compound generally obtained from plant and animal sources where it occurs in triglycerides, esters of glycerol with long-chain carboxylic acids. Glycerin has the chemical formula C₃H₈O₃.

Caprylic/capric triglyceride refers to fractionated coconut oil.

Coconut oil is an oil derived from the kernels, meat, and milk of the coconut palm fruit. Coconut oil is a white solid fat below around 25° C. (77° F.), and a clear thin liquid oil in warmer climates.

Olive oil is a liquid fat obtained by pressing whole olives, the fruit of Olea europaea, and extracting the oil. It includes mainly oleic acid (up to 83%), with smaller amounts of other fatty acids including linoleic acid (up to 21%) and palmitic acid (up to 20%).

Vegetable oil is a type of oil extracted from seeds or from other parts of edible plants. Like animal fats, vegetable fats are mixtures of triglycerides. Suitable examples of vegetable oils include (but are not limited to) soybean oil, corn oil, grape seed oil, cocoa butter, palm oil, rice bran oil, almond oil, avocado oil, canola oil, cottonseed oil, peanut oil, and safflower oil.

Dimethicone (also known as polydimethylsiloxane ((PDMS)) or dimethylpolysiloxane) is a silicone polymer with a wide variety of uses. Dimethicone has the chemical formula CH₃[Si(CH₃)₂O]ₙSi(CH₃)₃, where n is the number of repeating monomer [Si(CH₃)₂O] units.

Steric acid is a saturated fatty acid with an 18-carbon chain. Steric acid has the formula CH₃ (CH₂)₁₆CO₂H. Stearic acid is a prevalent fatty acid in nature, found in many animal and vegetable fats.

Butylene glycol (also referred to as butanediol) can refer any one of the four stable structural isomers 1,2-butanediol, 1,3-butanediol, 1,4-butanediol, and 2,3-butanediol.

Cetyl alcohol (also known as hexadecan-1-ol and palmityl alcohol) is a C-16 fatty alcohol with the formula CH₃ (CH₂)₁₅OH.

The moisturizing base can be present in the disclosed composition in an amount of about 0-98 weight percent. Thus, the disclosed composition can include moisturizing base in an amount of at least about (or no more than about) 0, 1, 5, 10, 15, 20, 25, 30, 35, 40, 45, 50, 55, 60, 65, 70, 75, 80, 85, 90, 95, or 98 weight percent, based on the total weight of the composition.

The composition can also optionally include one or more additional materials. For example, the composition include a solvent in an amount of about 1-50 weight percent (e.g., at least/no more than about 0, 1, 5, 10, 15, 20, 25, 30, 35, 40, 45, or 50 weight percent). The term “solvent” includes any material capable of dissolving one or more sulfated polysaccharides to form a uniformly dispersed mixture. In some instances, the solvent can be selected from water or an organic solvent. Exemplary organic solvents include (but are not limited to) petroleum ether, cyclohexane, toluene, carbon tetrachloride, dichloromethane, chloroform, diethyl ether, diisopropyl ether, ethyl acetate, butanol, n-propanol, ethanol, methanol, polyethylene glycol, propylene glycol, and pyridine. In some instances, the solvent is a glycol (e.g., butylene glycol, caprylyl glycol, and/or propylene glycol).

The disclosed compositions may comprise one or more additional ingredients selected from, for example, moisturizing agents, diluents, fragrances, pH adjusters, emulsifiers, preservatives, antioxidants, emollients, surfactant agents, solubilizing agents, viscosity modifiers, and/or chelating agents. Each additional ingredient can be present in an amount of from about 0.1-10 weight percent (e.g., at least/no more than about 0.1, 0.5, 1, 1.5, 2, 2.5, 3, 3.5, 4, 4.5, 5, 5.5, 6, 6.5, 7, 7.5, 8, 8.5, 9, 9.5, or 10 weight percent), based on the total weight of the composition.

Non-limiting examples of moisturizing agents that can be used with the disclosed compositions include amino acids, chondroitin sulfate, diglycerin, erythritol, fructose, glucose, glycerin, glycerol polymers, glycol, 1,2,6-hexanetriol, honey, hyaluronic acid, hydrogenated honey, hydrogenated starch hydrolysate, inositol, lactitol, maltitol, maltose, mannitol, natural moisturizing factor, PEG-15 butanediol, polyglyceryl sorbitol, salts of pyrollidone carboxylic acid, potassium PCA, propylene glycol, sodium glucuronate, sodium PCA, sorbitol, sucrose, trehalose, urea, and xylitol.

Non-limiting examples of diluents include water, saline, cellulose, dicalcium phosphate, mannitol, lactose, starch, Ringer's solution, bicarbonate solutions, dextrose solutions, gelatin, vegetable gum, talc, vegetable oil, glycol, and combinations thereof. The term “diluent” therefore broadly includes any chemical compounds that are used to dilute a compound of interest prior to delivery. Diluents can also serve to stabilize compounds.

Non-limiting examples of fragrances include aldehydes, ketones, acetals, ketals acids, esters, alcohols, lactones, nitriles, ethers, acetates, hydrocarbons, sulfur-nitrogen- and oxygen-containing heterocyclic, polycyclic and macrocyclic compounds, silicic esters, and mixtures thereof. Any fragrance can be used.

Non-limiting examples of pH adjusters include any suitable acid or base, such as one or more of hydrochloric acid, sulfuric acid, nitric acid, boric acid, carbonic acid, bicarbonic acid, gluconic acid, sodium hydroxide, potassium hydroxide, aqueous ammonia, citric acid, monoethanolamine, lactic acid, acetic acid, succinic acid, fumaric acid, maleic acid, phosphoric acid, methanesulfonic acid, malic acid, propionic acid, trifluoroacetic acid, sodium formate, potassium formate, sodium acetate, potassium acetate, sodium citrate, potassium citrate, sodium glycolate, potassium glycolate, sodium maleate, potassium maleate, sodium phosphate, potassium phosphate.

Non-limiting examples of emulsifiers include one or more of alkyl phosphates, glyceryl oleate citrates, glyceryl monostearate, propylene glycol monostearate, and sodium stearoyllactylate, cetyl betaine, glycolether glycerine, glycereyl stearate citrates, stearic acid esters, sorbitan esters, ethoxylated sorbitan esters, ethoxylated mono-, di- and tri glycerides, methyl glucose esters, and mixtures thereof. Suitable emulsifiers can therefore include any additive that creates an emulsion, a dispersion of one immiscible liquid into another, by reducing the interfacial tension between the two liquids to achieve stability.

Non-limiting examples of preservatives include phenol, m-cresol, benzyl alcohol, chlorobutanol, ethanol, phenoxyethanol, p-chlor-m-cresol, methyl paraben, propyl paraben, benzalkonium chloride, and thiomersal. Suitable preservatives can therefore include any known pharmaceutically acceptable preservative that functions by inhibiting bacteria, fungi, yeast, mold, other microbe, and/or by inhibiting oxidation.

Non-limiting examples of antioxidants include one or more of niacinamide, vitamin E, Coenzyme Q10, idebenone, lycopene, green tea polyphenols, silybin, resveratrol, grape seed extract, Oregon grape root (Mahonia aquifolium) extract, pomegranate extract, genistein, pycnogenol, curcumin, curcuminoids, or combinations thereof. In yet another embodiment, a composition comprises niacinamide, butylene glycol, tetrahexyldecyl ascorbate, caprylic/capric triglycerides, polyacrylate-13, cetyl ethylhexanoate, phenoxyethanol, hexylresorcinol, ethyl linoleate, polyisobutene, 4-ethoxybenzaldehyde, squalene, tocopherol, potassium sorbate, retinol, polysorbate 20, ethylhexylglycerine, phytic acid, disodium EDTA, Dunaliella salina extract. Suitable antioxidants can therefore include a component that may prevent and/or inhibit the formation of unacceptable amounts of oxidative degradants in the composition after a certain period of shelf life.

Non-limiting examples of emollients include sorbitol, glycol, diethylene glycol, methylene glycol, polypropylene glycol, polyethylene glycol, cetearyl octanoate, cyclomethicone, methyl palmitate, methyl stearate, isopropyl laurate, isopropyl myristate, isopropyl palmitate, ethylhexyl palmitate, lauryl lactate and cetyl lactate, propylene glycol, octyldodecanol, glycerol, diisopropyl adipate, and isostearic acid. The term “emollient” can therefore include any agent that can be used for the prevention or relief of dryness, as well as for the protection of the skin.

Non-limiting examples of surfactant agents include sodium laureth sulfate, cocamide diethanolamine, lauryl betaine, polyoxyethylene sorbitan monolaurate, polyoxyethylene sorbitan' monopalmitate, polyoxyethylene sorbitan monostearate, polyoxyethylene sorbitan monooleate, imidazoline cationic quaternary, phenylisooctyl polyethoxy ethanol, nonyl phenoxy polyethoxy ethanol, methyldodecylbenzyl trimethyl ammonium chloride and bis (trimethyl ammonium chloride) methyldodecyl xylene, sodium octyl sulfonate, and mixtures thereof. Thus, the term “surfactant agent” broadly includes any compound generally recognized in the art as having surface active qualities, such as anionic, cationic, nonionic, and zwitterionic compounds.

Non-limiting examples of solubilizing agents include water, monohydric or polyhydric alcohols, ether alcohols and mixtures thereof, selected from citric acid, acetic acid, succinic acid, maleic acid, benzoic acid, lactic acid, tartaric acid, barbituric acid, protocatechuic acid, gallic acid, 5-nitro-2-furoic acid, alpha-keto acids, hydrocarbons, dimethylether, polyoxyalkylene glycol ethers, amides, ketones, nitriles, chlorocarbons, esters, lactones, aryl ethers, fluoroethers, and 1,1,1-trifluoroalkanes, polyethylene glycol, glycerin, D-mannitol, trehalose, benzyl benzoate, ethanol, trisaminomethane, cholesterol, triethanolamine, sodium carbonate, sodium citrate, sodium salicylate, sodium acetate, and mixtures thereof. Thus, suitable solubilizing agents include any component that enhances the dissolution of one or more materials (e.g., sulfated polysaccharide) in an aqueous solvent.

Non-limiting examples of viscosity modifiers include guar gum, aliphatic polyester, aromatic polyester, polypropylene, mannan, polyvinyl alcohol, hectorite clay, garamite clay, organomodified clay, xanthan gum, pectin, cellulose, hydroxypropyl methylcellulose, poloxamers, and combinations thereof. Thus, suitable viscosity modifiers can include any material that can reduce or increase the viscosity of the disclosed compositions.

Non-limiting examples of f chelating agents include one or more ethylenediaminetetraacetic acid (EDTA), methyl glycine diacetic acid trisodium salt (MGDA), ethylene glycol tetraacetic acid (EGTA), diethylenetriamine pentaacetate (DTPA), diethylenetriaminepenta(methylenephosphonic) acid (DTPMP), hydroxyethylidene diphosphonic acid (HEDP), and mixtures thereof. Thus, suitable chelating agents can include any compound that can form stable complexes with metal ions by coordinating with them through multiple sites.

Non-limiting examples of antioxidants that can be used with the disclosed compositions include acetyl cysteine, ascorbic acid polypeptide, ascorbyl dipalmitate, ascorbyl methylsilanol pectinate, ascorbyl palmitate, ascorbyl stearate, BHA, BHT, t-butyl hydroquinone, cysteine, cysteine HCl, diamylhydroquinone, di-t-butylhydroquinone, dicetyl thiodipropionate, dioleyl tocopheryl methylsilanol, disodium ascorbyl sulfate, distearyl thiodipropionate, ditridecyl thiodipropionate, dodecyl gallate, erythorbic acid, esters of ascorbic acid, ethyl ferulate, ferulic acid, gallic acid esters, hydroquinone, isooctyl thioglycolate, kojic acid, magnesium ascorbate, magnesium ascorbyl phosphate, methylsilanol ascorbate, natural botanical anti-oxidants such as green tea or grape seed extracts, nordihydroguaiaretic acid, octyl gallate, phenylthioglycolic acid, potassium ascorbyl tocopheryl phosphate, potassium sulfite, propyl gallate, quinones, rosmarinic acid, sodium ascorbate, sodium bisulfite, sodium erythorbate, sodium metabisulfite, sodium sulfite, superoxide dismutase, sodium thioglycolate, sorbityl furfural, thiodiglycol, thiodiglycolamide, thiodiglycolic acid, thioglycolic acid, thiolactic acid, thiosalicylic acid, tocophereth-5, tocophereth-10, tocophereth-12, tocophereth-18, tocophereth-50, tocopherol, tocophersolan, tocopheryl acetate, tocopheryl linoleate, tocopheryl nicotinate, tocopheryl succinate, and tris(nonylphenyl)phosphite.

In use, the disclosed composition can be used to heal, conceal, and/or moisturize a bruise. Particularly, the composition can be topically applied to a bruise using any suitable mechanism, such as the hands, fingers, a sponge pad, spot applicator, or combinations thereof. The composition can then be allowed to dry, whereby the bruise is concealed (e.g., the bruise is fully or partially visually undetectable) due to the colored material. For example, the bruise can be about 10-100% undetectable (at least/no more than about 10, 20, 30, 40, 50, 60, 70, 80, 90, or 100 percent). Healing of the bruise is also promoted due to the noted bruise healing materials using any of a wide variety of mechanisms, such as (but not limited to) stimulating blood flow to the bruised area, reducing swelling to the bruise, reducing inflammation to the bruised area of skin, strengthening blood vessels in the bruised area, and the like. In some embodiments, the bruised area of skin is also moisturized using the noted moisturizing materials.

Thus, the composition can be a topical composition. The term “topical” refers to administration of the composition at or immediately beneath the point of application. For example, a topical composition can be applied directly to body surfaces such as the skin to treat a desired condition. The disclosed composition can have any suitable form, such as an ointment, cream, lotion, gel, paste, serum, emulsion, powder, solution, etc.

In some embodiments, the composition is color transfer resistant, such that the composition does not bleed and is not transferred upon contact with clothing or other items.

In some embodiments, the composition has a pH of about 4-9 (e.g., 4, 5, 6, 7, 8, or 9 (or any value therebetween)).

The healing time for the bruise upon application of the disclosed topical compositions may be based on each individual subject. For example, the older the subject is, the longer the healing time may be. In addition, the nutritional state of the subject, application frequency of the disclosed compositions, and general state of the health of the subject may also affect the rate of healing. Thus, the disclosed compositions can be used with various treatment regimens. In some instances, the topical compositions can be administered 1, 2, 3, or more times per day to the bruised area of the skin. In some embodiments, the topical compositions described herein are administered for at least 1 day, 2 days, 3 days, 4 days, 5 days, 6 days, 1 week, 2 weeks, or more to improve a bruise. In other embodiments, the disclosed composition may be applied as a maintenance therapy, wherein the composition is continuously applied as needed or applied on a scheduled basis over time while the subject needs such treatment (e.g., until the bruise disappears).

The term “improves” is used to convey that the application of the disclosed compositions changes the appearance, form, characteristics, and/or the physical attributes of the tissue to which it is being provided, applied, or administered. The change in form may be demonstrated by an enhanced appearance of the skin, decrease in vascular extravasation into the skin, decrease in cutaneous petechiae, purpura or ecchymoses, decrease in pigmentation, and/or hastening the resolution of the purpuric/hemorrhagic skin lesions in a subject. Thus, during the treatment period, the bruise is disaggregated, the discoloration is significantly decreased or disappears, the pain associated with the bruise is significantly decreased or disappears, and the inflammation is diminished in some embodiments.

The disclosed compositions can be applied by any method. For example, in some embodiments, the compositions may be applied by hand by the subject or another person, such as a clinician. In other embodiments, the compositions may be packaged with an applicator (such as a wand, swath of cloth, or applicator pad).

The term “subject” broadly refers to the target of administration, e.g., an animal. Thus, the subject of the disclosed methods can be a human, non-human primate, horse, pig, rabbit, dog, sheep, goat, cow, cat, guinea pig, rodent, etc. The term does not denote a particular age or sex. Thus, adult and newborn subjects of any age are intended to be covered. In some embodiments, the subject is a mammal.

As a result of treatment with the disclosed compositions, the body can more quickly invade the injured tissue with white blood cells that begin the breakdown and reabsorption process to remove the trapped clotted blood and break it down into biliverdin and bilirubin. The visible color of the bruise can then transition toward green and then brownish in color.

Thus, the entire blood reabsorption and tissue rebuilding processes underpinning bruising and its healing process can be greatly enhanced if the applied composition can improve blood microcirculation and aid in lymphatic drainage. Additionally, if the ingredient also has a mild anti-coagulant activity it may also aid the healing process. Additionally, combining the noted sulfated polysaccharides with conventional topical bruising agents as mentioned above enhances the ability of the body to reabsorb blood via microcirculation resulting from a contusion.

Thus, compositions as described herein improve bruising by improving healing or appearance of the bruise. In some embodiments, the compositions improve bruising by accelerating resolution of the bruise. For example, the compositions accelerate the transition of blue coloration to red coloration of the bruise. In some embodiments, improved appearance of the bruise comprises a reduced size of the bruise. In some embodiments, improved appearance of the bruise comprises reduced discoloration of the skin. In some embodiments, improved appearance of the bruise comprises reduced swelling.

In some embodiments, the disclosed compositions improve bruising by at least or about 10%, 15%, 20%, 25%, 30%, 35%, 40%, 45%, 50%, 55%, 60%, 65%, 70%, 75%, 80%, 85%, 90%, 95%, or more than 95% compared to bruises not treated with the disclosed compositions.

In some embodiments, the disclosed compositions improve bruising by at least about 0.5×, 1.0×, 1.5×, 2.0×, 2.5×, 3.0×, 3.5×, 4.0×, 5.0×, 6.0×, 7.0×, 8.0×, 9.0×, 10×, or more than 10× compared to bruises not treated with the disclosed compositions. In some embodiments, the bruising is improved at least 1 day or 2 days (e.g., about 2 weeks) after administering the topical composition. Thus, the term “improved” is used to convey that use of the disclosed composition changes either the characteristics and/or the physical attributes of the tissue to which it is being provided, applied, or administered. The term “improved” may also be used in conjunction with a tissue state such that when a tissue state is “improved” the symptoms or physical characteristics associated with the state (e.g., the bruised state) are diminished, reduced, or eliminated.

In some embodiments, a bruise or purpura treated with the disclosed composition is fully or partially concealed (e.g., at least/no more than about 50, 60, 70, 80, 90, or 100 percent concealed). 100 percent concealed indicates that the bruise or purpura is not visible on the skin.

The presently disclosed subject matter includes many benefits over prior art wound healing/concealing compositions. For example, the composition is simple to use, such that even the elderly or children can easily heal, moisturize, and conceal bruises.

The composition can be used in one step to effectively heal, moisturize, and conceal a bruise on a user's skin. Beneficially, multiple steps are not required, adding to the ease of use.

The disclosed composition partially or fully conceals a bruise on a user's skin, while also providing wound healing and/or moisturizing characteristics to the skin.

Further, the disclosed composition is not overly tinted such that it appears as moisturizer when applied to a user's skin at least once a day.

A single composition can be used to treat several different skin types and skin tones, thereby simplifying use.

The composition is also compatible with conventional makeup foundations.

Continuing, the composition is transfer resistant, prevent bleeding onto clothing and other materials upon contact.

Although the present invention and its advantages have been described in detail, various changes, substitutions and alterations can be made herein without departing from the spirit and scope of the invention as defined by the appended claims. Moreover, the scope of the present application is not intended to be limited to the embodiments of the process, machine, manufacture, composition of matter, means, methods and steps described in the specification. As one of ordinary skill in the art will readily appreciate from the disclosure of the present invention, processes, machines, manufacture, compositions of matter, means, methods, or steps, presently existing or later to be developed that perform substantially the same function or achieve substantially the same result as the corresponding embodiments described herein may be utilized according to the present invention. Accordingly, the appended claims are intended to include within their scope such processes, machines, manufacture, compositions of matter, means, methods, or steps.

### EXAMPLES

The following Examples provide illustrative embodiments of the presently disclosed subject matter. Considering the present disclosure and the general level of skill in the art, those of ordinary skill can appreciate that the following Examples are intended to be exemplary only and that numerous changes, modifications, and alterations can be employed without departing from the scope of the presently disclosed subject matter.

### Example 1

### Formulation of the Composition

A moisturizing formula was selected comprising water, glycerin, and caprylic and capric triglycerides (MCT Oil). The moisturizing formula was beneficially stable, highly moisturizing, and was believed to integrate and effectively deposit silica, titanium dioxides, colored micas, chalk and/or colored iron oxides.

It was next determined which color tint(s) provide the best coverage for the type of bruise needed to conceal. The color tint(s) were selected to be an acceptable color for a moisturizer and applicable across several skin types. It is noted that bruises go through various phases of healing that are associated with different colors. Bruises generally start red, then turn blue and/or purple, and then finally brown before they clear from the skin. The longest phases are the purple and brown colors lasting possibly weeks before they fully clear from the skin. Using the “color wheel”, yellow was selected to counter (and cancel out) purple and to some extent brown-colored bruises. It is noted that a pink tinted formula can be used to cancel out brown in a bruise. Further, yellow and pink tints can also be combined for use in the disclosed formulation.

The level and intensity of the colored materials was tailored to properly accommodate the skin color type (Fitzpatrick Phototype). A single formula that partially to fully visually concealed bruises was created for use on phototypes 1, 2 and possibly 3. Accordingly, the level of the tint was gradually increased. White titanium dioxide was used in combination with yellow and/or pink (red) oxides. Different levels and multiple applications on bruised skin were tested.

### Example 2

### Application of the Composition

The composition of Example 1 was applied to a bruise on the skin of a user and only the immediate surrounding skin. It was not applied to the entire arm or leg area. A 4-5 day old bruise on the upper thigh of a user was selected for treatment with the composition.

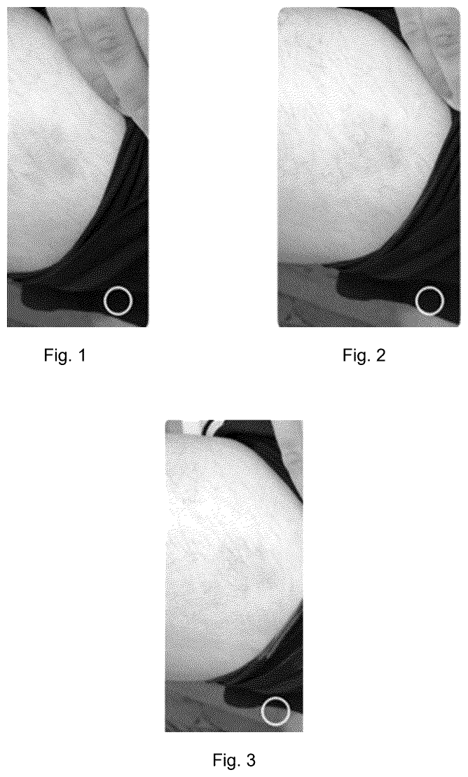

FIG. 1 is a photo of the untreated bruise. FIG. 2 is a photo of the same bruise after a single light application of the composition.

FIG. 3 illustrates the same bruise with a second light treatment of the composition applied.

After light topical application of the composition, it was observed that the first and lowest level of the colored materials did not visibly conceal the bruise, even after two applications on Phototype II Skin, as illustrated in the figures.

However, when the composition was formulated at twice the level, it was visibly observed that the bruise on the upper thigh was partially concealed. More so, with a second application. It was noted that the bruise could be mostly or fully concealed if a higher amount of the tinted moisturizer on the bruise was applied.

### Example 3

### Use of the Composition to Conceal a Bruise

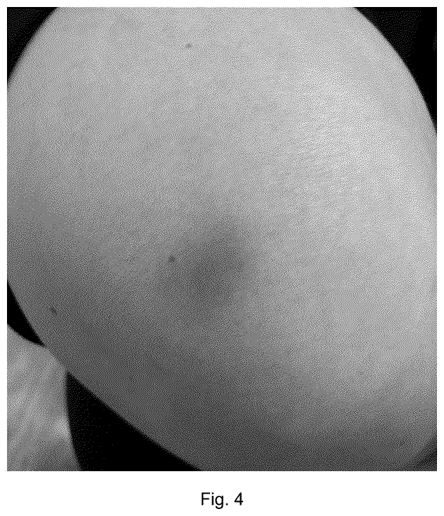

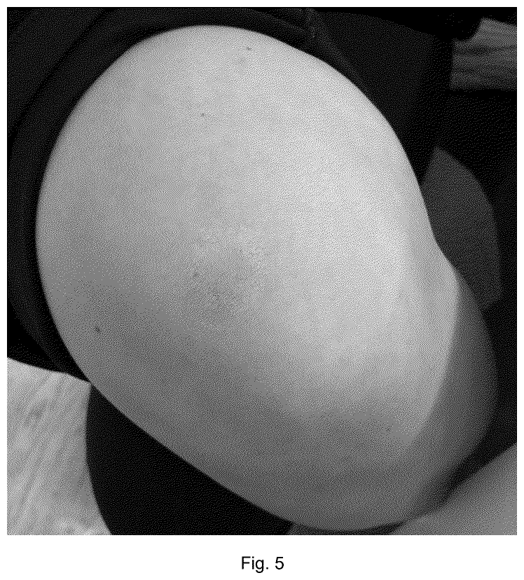

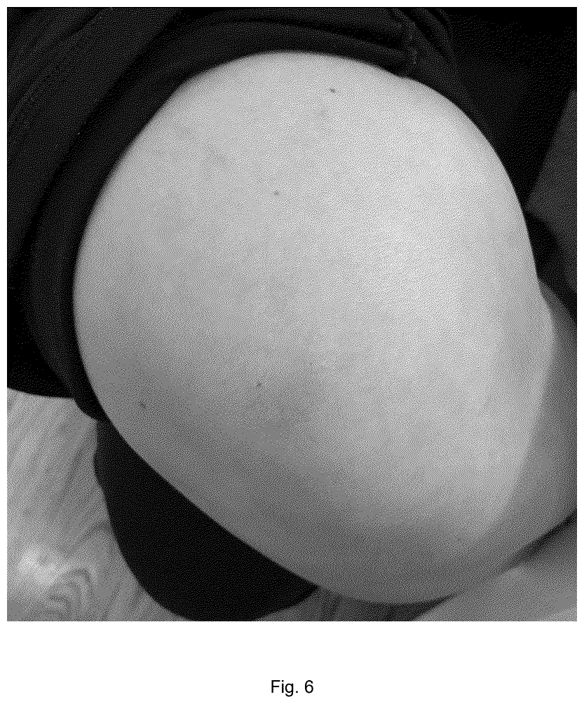

The composition of Example 1 was applied over a large bruise on a subject that was purple, pink, and red in color. FIG. 4 illustrates the bruise prior to treatment. An amount of the composition was applied over the entire colored region of the bruise, extending to the surrounding skin, as illustrated in FIG. 5. After the composition dried (as shown in FIG. 6), the bruise was substantially less visible. The disclosed composition effectively concealed the bruise on the skin of the subject, while also providing active ingredients that promote healing of the bruise.

What is claimed is:

1. A composition comprising:
one or more skin safe colored materials that deposit on the skin and partially to fully color conceal, color correct, or both color conceal and color correct bruises, senile purpura, solar purpura, or combinations thereof in various healing phases; and
one or more active bruise healing materials.

2. The composition of claim 1 further comprising a moisturizing base.

3. The composition of claim 1:
wherein the skin safe colored materials are selected from: titanium dioxide, colored iron oxides, mica, chalks, clays, or combinations thereof; and
wherein the bruise healing materials are selected from: Arnica, Vitamin K, Vitamin C, Vitamin E, Tranexamic acid, retinol, bakuchiol, retinyl palmitate, or combinations thereof.

4. The composition of claim 2, wherein the moisturizing base comprises glycerin, caprylic triglycerides, capric triglycerides, coconut oil, olive oil, vegetable oils, dimethicone, steric acid, butylene glycol, cetyl alcohol, or combinations thereof.

5. The composition of claim 1, wherein the skin safe colored materials comprise a yellow, brown, or pink tint that is configured to visually conceal or color correct a bruise partially or fully without the need of an additional colored material.

6. The composition of claim 3, wherein the skin safe colored materials are selected from about 1-99 weight percent of titanium dioxide, 1-99 weight percent of yellow iron oxide, 1-99 weight percent of pink iron oxides, 1-99 weight percent of red iron oxides, 1-99 weight percent of mica, 1-99 weight percent chalk, and 1-99 weight percent clay.

7. The composition of claim 1, wherein the composition is configured to partially or fully conceal a skin bruise after one or more applications.

8. The composition of claim 1, wherein the composition is color transfer resistant.

9. The composition of claim 1, whereby the composition is compatible and covered using a skin color matched foundation or makeup.

10. The composition of claim 1, further comprising one or more depigmenting materials.

11. The composition of claim 1, further comprising one or more pain reducing or relieving ingredients.

12. A method of concealing a bruise using the composition of claim 1, the method comprising:
applying the composition to a bruise;
allowing the composition to dry;
whereby the bruise is concealed and healing is promoted.

13. The method of claim 12, wherein the applying is facilitated using a sponge pad, a spot applicator, or both.
